(19) United States
(12) Patent Application Publication
(10) Pub. No.: US 2010/0008872 A1
(43) Pub. Date: Jan. 14, 2010

(54) SILICA COATED ZINC OXIDE PARTICLES OBTAINABLE BY A FLAME PYROLYSIS PROCESS

(75) Inventors: Stipan Katusic, Bad Soden (DE); Sven Hill, Aschaffenburg (DE); Michael Kroell, Linsengericht (DE); Guido Zimmermann, Bruehl (DE); Peter Kress, Karlstein (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: EVONIK DEGUSSA GmbH, Essen (DE)

(21) Appl. No.: 12/375,062

(22) PCT Filed: Jun. 26, 2007
(86) PCT No.: PCT/EP07/56371

§ 371 Date: Jan. 26, 2009

(30) Foreign Application Priority Data
Aug. 17, 2006 (DE) 10 2006 038 518.7

(51) Int. Cl. A61K 8/27; B32B 15/02; C01B 33/20; A61Q 17/04

(52) U.S. Cl. 424/59; 428/405; 423/326

(57) ABSTRACT

Zinc silicon oxide particles with a core-shell structure which have a Zn/Si ratio of from 2 to 75, in atom %/atom %, whose fraction of Zn, Si and O is at least 99% by weight, based on the zinc silicon oxide particles, which have a BET surface area of from 10 to 60 m²/g, a weight-averaged primary particle diameter of from 10 to 75 nm an average aggregate area of less than 40 000 nm² and an average aggregate diameter (ECD) of less than 200 nm, whose core is crystalline and consists of aggregated primary particles of zinc oxide and whose shell surrounds the aggregated zinc oxide primary particles and consists of one or more compounds containing the elements Si and O. Dispersion, coating composition and sunscreen formulation comprise the zinc silicon oxide particles.

The invention relates to coated zinc oxide particles and to their preparation. The invention also relates to a dispersion comprising these zinc oxide particles, to a coating composition and to a sunscreen formulation.

It is known, in order to reduce the photocatalytic activity of UV-absorbing substances, to provide these with an inert shell. The UV-absorbing substances may be organic or inorganic substances. Of particular significance are titanium dioxides and zinc oxides coated with silicon dioxide since these are used in sunscreen formulations.

Zinc oxides coated with silicon dioxide are obtained according to the prior art in a process which generally involves the steps of preparing zinc oxide powder, preparing a dispersion comprising this powder, adding a source of silicon and a base to this dispersion, separating off and purifying the powder obtained and subsequent thermal treatment.

EP-A-988853 discloses zinc oxide coated with silicon dioxide. For the preparation, zinc oxide particles are initially introduced into a mixture of water and an organic solvent, and alkali and a tetraethoxysilane are added. A powder is obtained which has a relatively low surface functionality and a high degree of intergrowth of the particles. As a result, firstly incorporation of the particles into a cosmetic formulation is hindered, and secondly their stability with regard to sedimentation is limited.

EP-A-1284277 discloses a similar process to that in EP-A-988853, but dispenses with the use of organic solvents. The resulting zinc oxide powder coated with silicon dioxide has only a slight degree of intergrowth.

A disadvantage of the processes in which the shell is applied from a liquid phase is the reproducibility since the structure of the powders used in the liquid phase is critically dependent on the solvents used and the pH. Furthermore, the powders can be contaminated by the precipitation process, for example by incorporated alkali.

Besides the methods in which the shell is applied from a liquid medium, there also exist methods in which coated particles are prepared in gas-phase reactions.

U.S. Pat. No. 5,268,337 describes the preparation of titanium dioxide particles coated with silicon dioxide in a flame-hydrolytic process.

WO 96/36941 discloses the gas-phase oxidation of a thermally decomposable titanium dioxide precursor and a thermally decomposable silicon dioxide precursor with oxygen in a tubular reactor at temperatures of at least 1300° C.

WO 2004056927 discloses a process in which titanium dioxide coated with silicon dioxide is obtained by means of flame hydrolysis. Here, a vaporizable silicon compound, usually silicon tetrachloride, and a vaporizable titanium compound, usually titanium tetrachloride, is combusted in a hydrogen/oxygen flame under certain conditions. The powder obtained consists of aggregated primary particles which have a shell made of silicon dioxide and a core made of titanium dioxide.

The gas-phase reactions are restricted to the preparation of coated titanium dioxides. The preparation of zinc oxides coated with silicon dioxide by such a process is not yet described.

The products produced according to the outlined process usually have good properties with regard to their UV absorption, photocatalytic properties and transparency. Nevertheless, there is a desire in particular for sunscreen formulations and coating compositions which, with regard to UV absorption, photocatalytic activity and transparency, have improved values compared to the prior art. It was therefore an object of the invention to provide such a material.

It was a further object of the invention to provide a process for the preparation of this material which avoids the disadvantages of the prior art. The process should be easy to carry out and be economical.

The invention provides zinc silicon oxide particles with a core-shell structure, where
they have a Zn/Si ratio of from 2 to 75, in atom %/atom %,
the fraction of Zn, Si and O is at least 99% by weight, based on the zinc silicon oxide particles,
they have a
   BET surface area of from 10 to 60 m²/g,
   a weight-averaged primary particle diameter of from 10 to 75 nm
   an average aggregate area of less than 40 000 nm² and
   an average aggregate diameter (ECD) of less than 200 nm,
the core is crystalline and consists of aggregated primary particles of zinc oxide and
the shell surrounds the aggregated zinc oxide primary particles and consists of one or more compounds containing the elements Si and O.

The BET surface area of the zinc silicon oxide particles according to the invention is 10 to 60 m²/g. Preferably, they can have a BET surface area of from 20 to 40 m²/g and particularly preferably one of from 25 to 35 m²/g.

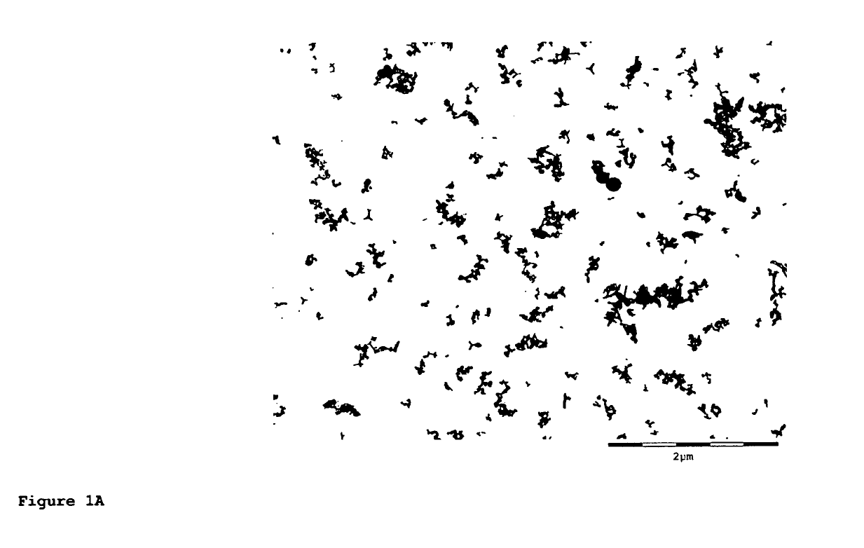

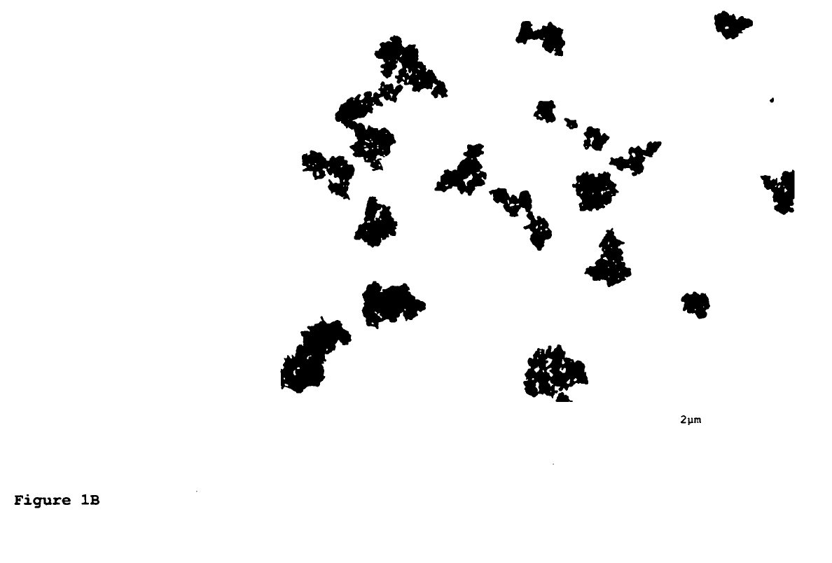

FIG. 1A shows a TEM micrograph of the zinc silicon oxide particles according to the invention. Image analysis is used to ascertain a weight-averaged primary particle diameter of the zinc silicon oxide particles according to the invention of from 10 to 75 nm, preferably 20 to 50 nm.

Image analysis is likewise used to determine the average aggregate area of the zinc silicon oxide particles according to the invention. Here, the zinc silicon oxide particles are characterized by a small average aggregate area of less than 40 000 nm². In a preferred embodiment, the average aggregate area is less than 20 000 nm², where a range from 1000 to 10 000 nm² may be particularly preferred.

The average aggregate diameter (ECD), likewise determined by means of image analysis, of the zinc silicon oxide particles according to the invention is smaller than 200 nm. Preferably, the average aggregate diameter (ECD) is 50 to 150 nm.

The distances between the lattice planes, determined from HR-TEM micrographs, show that the core of the zinc silicon oxide particles according to the invention consists of crystalline zinc oxide.

The shell comprises, according to XPS-ESCA analysis (XPS=X-ray photoelectron spectroscopy; ESCA=electron spectroscopy for chemical analysis) and TEM-EDX analysis (transmission electron microscopy [TEM] in conjunction with energy-dispersive analysis of characteristic X-rays [EDX]), compounds which contain the elements Si and O. Furthermore, the shell can also have compounds containing zinc.

The amount of these elements and of the corresponding compounds in the shell cannot be determined exactly. However, evaluation of TEM-EDX and XPS-ESCA spectra clearly shows that Si and O are the main components of the shell, and Zn, if present, is only present in secondary amounts. The presence of zinc in the shell does not alter the properties of the zinc silicon oxide particles according to the invention.

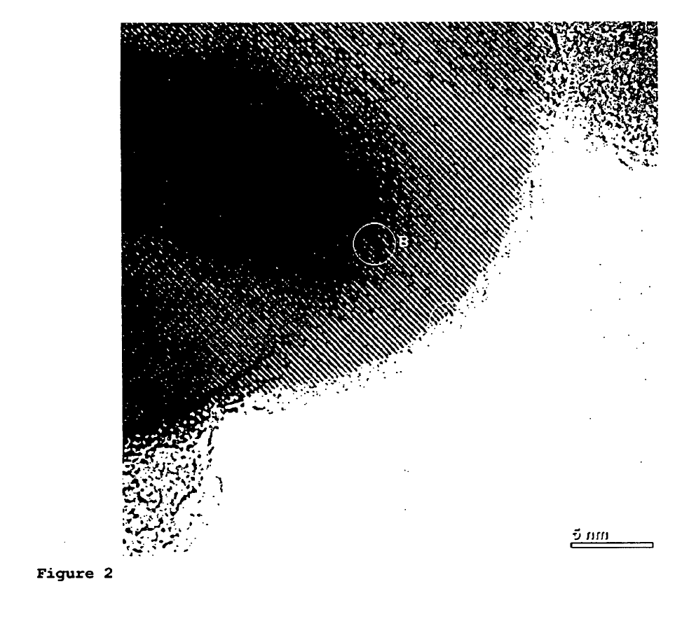

FIG. 2 shows a high-resolution TEM micrograph which clearly shows the shell of the zinc silicon oxide particles according to the invention.

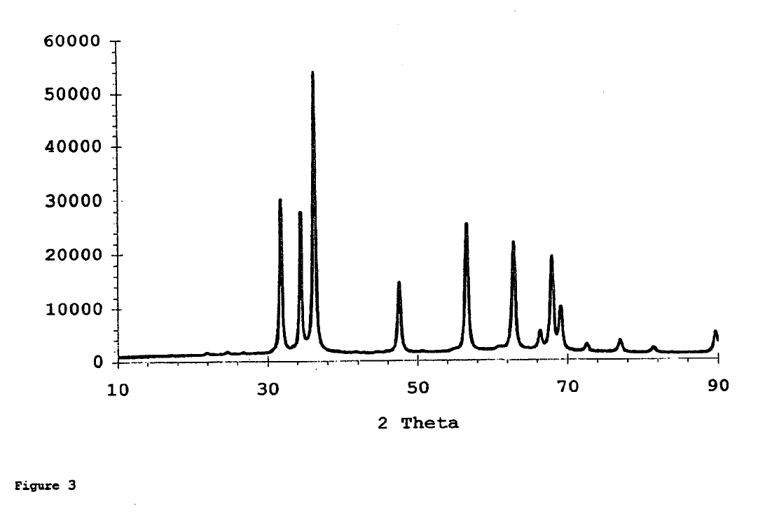

The shell of the zinc silicon oxide particles according to the invention is preferably in amorphous form. In addition, the shell can have small crystalline fractions which can be detected by X-ray diffractometry. The amounts are usually barely above the detection limit in the X-ray diffractometry (FIG. 3).

The statement that the shell can have crystalline constituents is based firstly on the evaluation of the lattice distances in HR-TEM micrographs, which clearly identifies the core as zinc oxide, secondly the fact that, apart from zinc oxide, the X-ray diffractogram shows further signals of low intensity. The assignment of these signals to compounds is currently not possible. The crystalline constituents do not influence the properties of the zinc silicon oxide particles according to the invention.

The zinc silicon oxide particles according to the invention have a Zn/Si ratio of from 2 to 75. It has been shown that the zinc silicon oxide particles with a Zn/Si ratio of from 3 to 15, in particular 3.5 to 10, have particularly good properties in sunscreen formulations and coating compositions.

The thickness of the shell of the zinc silicon oxide particles according to the invention is not limited. A thicker coating layer is favourable for reducing the photocatalytic activity, but unfavourable for the UV absorption of the zinc silicon oxide particles. Zinc silicon oxide particles with a shell thickness of from 0.1 to 1.0 nm have favourable values for UV absorption and photocatalytic activity and are therefore preferred, for example, for applications in the field of sunscreen formulations. Particular preference is given to a range from 1 to 5 nm.

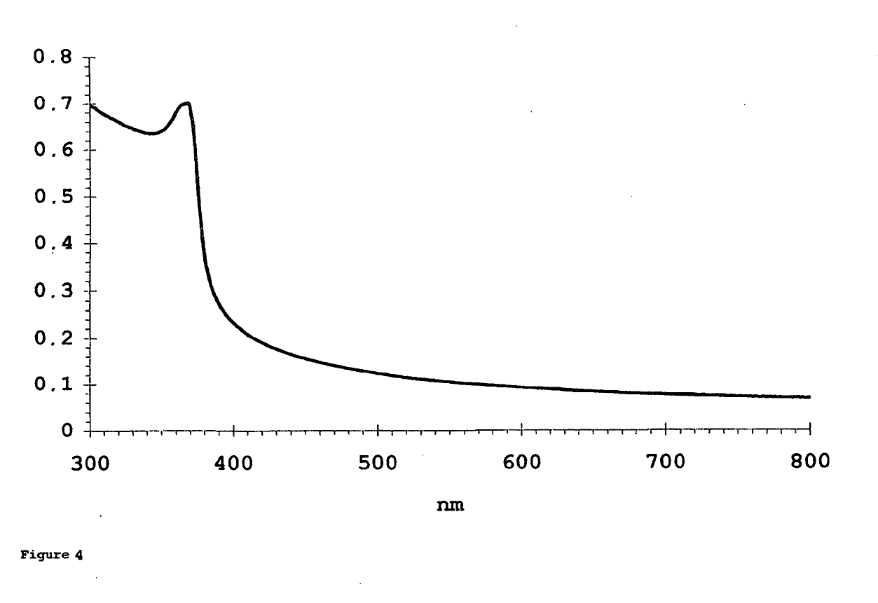

The zinc silicon oxide particles according to the invention preferably have a transparency, defined as the ratio of maximum absorbance/absorbance at 450 nm, of from 4 to 8. The wavelength of the maximum absorbance for the particles according to the invention can be 370±3 nm. FIG. 4 shows a UV spectrum of zinc silicon oxide particles according to the invention.

The zinc silicon oxide particles according to the invention advantageously have a photocatalytic activity, expressed by the photon efficiency and determined by the degradation of dichloroacetic acid (DCA) as model noxious substance, which is less than 0.4%, particularly preferably less than 0.2%. It is also possible to provide zinc silicon oxide particles according to the invention in which no degradation of DCA can be established, i.e. which are not photocatalytically active.

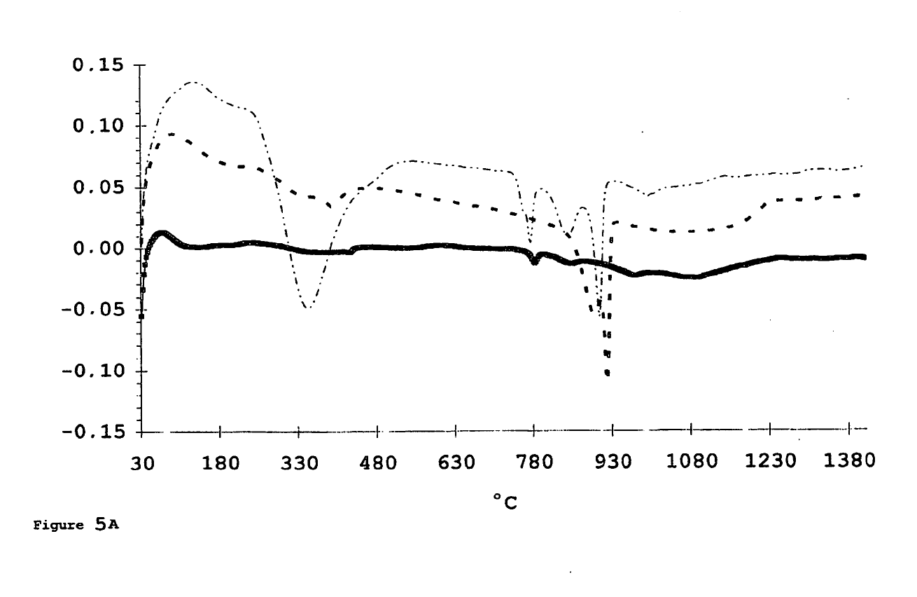

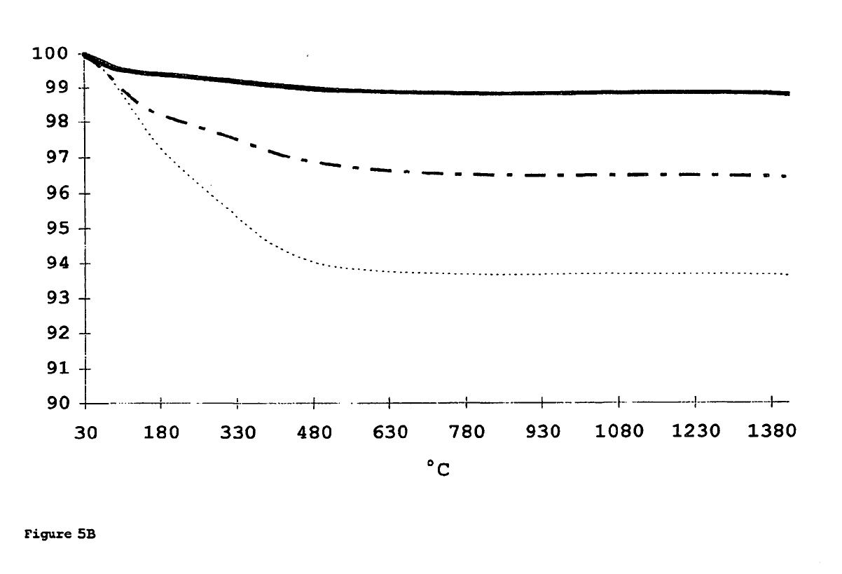

In one particular embodiment, the zinc silicon oxide particles according to the invention can be characterized in that, upon heating to 1400° C., they lose less than 2% of their mass, and phase conversions occur only to a slight extent (FIG. 5A and FIG. 5B). In this case, the heat of reaction between 700° C. and 1200° C. is less than 175 J/g.

The fraction of Zn, Si and O in the zinc silicon oxide particles according to the invention is, in total, at least 99% by weight. As a rule, the content is at least 99.5% by weight to 99.7% by weight.

If the zinc silicon oxide particles according to the invention are to be a constituent of a cosmetic or pharmaceutical preparation, the fractions of Pb can be at most 20 ppm, of As at most 3 ppm, of Cd at most 15 ppm, of Fe at most 200 ppm, of Sb at most 1 ppm and of mercury at most 1 ppm.

Moreover, the zinc silicon oxide particles according to the invention can have a carbon content of at most 0.2% by weight, which can be brought about through the use of carbon-containing feed materials and/or process control. If required, a carbon content of less than 250 ppm can be realized through appropriate selection of the feed materials and/or through process control.

The invention further provides a process for producing zinc silicon oxide particles in which a mixture of zinc vapour and a hydrogen-containing combustion gas is passed to an oxidation zone of a reactor, where it is reacted with an oxygen-containing gas and at least one organic silicon compound at temperatures of from 500 to 1500° C., then the reaction mixture is cooled and the pulverulent solid is separated off from gaseous substances, where
the silicon compound is chosen from at least one compound from the group comprising
R′ₓSi(OR)₄₋ₓ where R=Me, Et; R′=H, Me, Et; x=0-4,
R″ᵤMeᵥSiOSiMeᵥR″ᵤ where R″=H, Et; u=0, 1, 2; v=1, 2, 3; u+v=3;
R′″₄Si where R′″=H, Me, Et and/or cyclic polysiloxanes (R″″MeSiO)y where R″″=H, Me, Et, y=3-5,
the oxygen fraction of the oxygen-containing gas is at least sufficient to completely oxidize zinc, the organic silicon compound and the hydrogen
the average residence time of the reactants in the oxidation zone is 5 ms to 30 s, preferably 10 ms to 100 ms.

The process according to the invention can be carried out so that the zinc vapour can be provided by vaporizing zinc powder or a zinc melt in a nonoxidizing atmosphere, for example an inert gas or a hydrogen-containing combustion gas. The vaporization step here can be carried out in the same reactor as where the oxidation takes place or can be carried out in a separate vaporization unit.

Particular preference is given to an embodiment in which the zinc vapour is obtained from zinc powder in the presence of a hydrogen-containing combustion gas in the reactor in which the oxidation takes place.

The silicon compound can be introduced into the oxidation zone as vapour or as liquid in the form of fine droplets.

Particularly suitable silicon compounds are Si(OMe)₄, Si(OEt)₄, MeSi(OEt)₃, Me₂Si(OEt)₂, Me₃SiOEt, HMe₂SiOSiMe₂H, Me₃SiOSiMe₃ and the cyclic polysiloxanes (Me₂SiO)₃, (Me₂SiO)₄ and (Me₂SiO)₅. Of very particular suitability is tetraethoxysilane (Si(OEt)₄; TEOS).

Here, the silicon compound and the oxygen-containing gas can be introduced into the oxidation zone together or separately. It is advantageous to firstly introduce the oxygen-containing gas into the oxidation zone in order to partially or completely oxidize zinc vapour (zinc oxidation zone). Subsequently, the silicon compound is oxidized with the oxygen still present in the mixture and optionally the oxygen introduced as atomizing air for the silicon compound (silicon oxidation zone).

Preferably, the process according to the invention is carried out by providing the temperatures required for the oxidation and optionally vaporization by a flame which is formed by igniting a hydrogen-containing combustion gas with an oxygen-containing gas.

Suitable combustion gases may be hydrogen, methane, ethane, propane, natural gas, acetylene or mixtures of the abovementioned gases. Hydrogen is most suitable. The temperature required for vaporizing the starting materials can be provided by a suitable selection of the abovementioned gases and the oxygen content of the flame. Preferably, hydrogen or mixtures with hydrogen are used.

In the zinc oxidation zone, preferably 1<lambda=15 and particularly preferably 6=lambda=10.

In the silicon oxidation zone, preferably 1<lambda=15, preferably 4=lambda=10. The lambda value is defined as the quotient of the oxygen content of the oxygen-containing gas divided by the oxygen requirement which is required for the complete oxidation of the combustion gas, the organic zinc or silicon compounds, in each case in mol/h.

If the flame is also used for vaporizing zinc powder or a zinc melt, then, in the vaporization zone, lambda is preferably chosen so that 0.5=lambda=1, preferably 0.7=lambda=0.95.

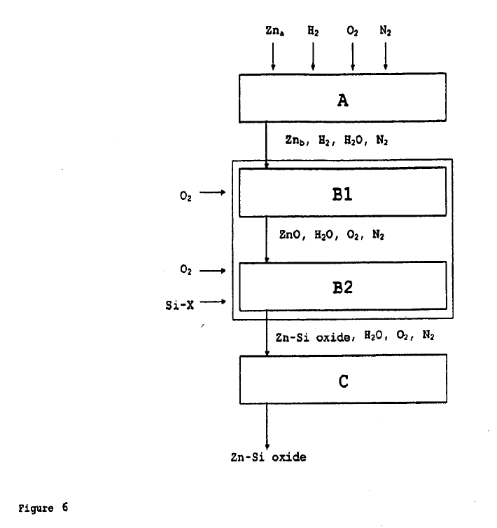

FIG. 6 shows a scheme of the preferred process according to the invention comprising A=vaporization zone, B1=zinc vapour oxidation zone, B2=silicon precursor SiX oxidation zone, C=quenching zone including filter unit. Here, Zna is a zinc powder or a zinc melt, Znb is zinc vapour. O₂ is air or another oxygen-containing gas.

A dispersion which comprises the particles according to the invention is further provided.

The liquid phase of the dispersion can be water, one or more organic solvents or an aqueous/organic combination where the phases are miscible.

Liquid organic phases can in particular be methanol, ethanol, n-propanol and isopropanol, butanol, octanol, cyclohexanol, acetone, butanone, cyclohexanone, ethyl acetate, glycolic ester, diethyl ether, dibutyl ether, anisole, dioxane, tetrahydrofuran, mono-, di-, tri- and polyglycol ether, ethylene glycol, diethylene glycol, propylene glycol, dimethylacetamide, dimethylformamide, pyridine, N-methylpyrrolidine, acetonitrile, sulpholane, dimethyl sulphoxide, nitrobenzene, dichloromethane, chloroform, tetrachloromethane, ethylene chloride, pentane, hexane, heptane and octane, cyclohexane, benzine, petroleum ether, methylcyclohexane, decalin, benzene, toluene and xylenes. Particularly preferred organic liquid phases are ethanol, n- and isopropanol, ethylene glycol, hexane, heptane, toluene and o-, m- and p-xylene.

Water is particularly preferred as liquid phase.

The dispersion according to the invention can also comprise pH regulators, surface-active additives and/or preservatives.

The content of zinc silicon oxide particles according to the invention can preferably be 0.5 to 60% by weight. Particular preference is given to an aqueous dispersion comprising 20 to 50% by weight, in particular 35 to 45% by weight, of the zinc silicon oxide particles according to the invention.

The pH of an aqueous dispersion according to the invention is preferably in a range from 6 to 9.

The average particle size in the dispersion can be varied within a wide range using appropriate dispersion units. These may be, for example, rotor stator machines, high-energy mills in which the particles are ground together through collision, planetary kneaders, stirred ball mills, ball mills operating as shaking unit, shaking plates, ultrasound units, roller mills or combinations of the abovementioned units.

A particularly small particle size can be obtained by using rotor stator machines and high-energy mills. The average particle size d₅₀ can here assume values of less than 180 nm, in particular less than 140 nm, determined by means of dynamic light scattering.

The invention further provides a coating preparation which comprises the zinc silicon oxide particles according to the invention or the dispersion according to the invention and at least one binder.

Suitable binders may be polyacrylates, polyurethanes, polyalkyds, polyepoxides, polysiloxanes, polyacrylo-nitrites and/or polyesters. In the case of dispersions which have one or more reactive thinners as liquid phase, an aliphatic urethane acrylate, for example Laromer® LR8987, BASF, can be particularly suitable as binder. The coating preparation according to the invention can particularly preferably comprise polyacrylates and/or polyurethanes.

The content of binder in the coating preparation is preferably between 0.1 and 50% by weight. A range between 1 and 10% by weight is particularly preferred.

The content of zinc silicon oxide particles in the coating preparation is preferably between 0.01 and 60% by weight. A range between 0.1 and 10% by weight is particularly preferred.

In addition, the coating preparation during the application can comprise compounds for changing the rheology of the coating preparation. Fillers containing silicon dioxide are particularly advantageous, with fumed silicon dioxide being particularly preferred. The amount can preferably be between 0.1 and 20% by weight, based on the total coating preparation.

In addition, the coating preparation can comprise organic solvents such as ethanol, butyl acetate, ethyl acetate, acetone, butanol, THF, alkanes or mixtures of two or more of these specified substances in amounts of from 1% by weight to 98% by weight, based on the total coating preparation.

The coating preparation according to the invention can be used for coating substrates made of wood, PVC, plastic, steel, aluminium, zinc, copper, MDF, glass, concrete.

The invention further provides a sunscreen formulation which comprises the zinc silicon oxide particles according to the invention.

The zinc silicon oxide particles according to the invention are present in the sunscreen formulation usually in an amount of from 0.5 to 20% by weight, preferably 1 to 10% by weight and particularly preferably 3 to 8% by weight.

Suitable chemical UV filters are all water-soluble or oil-soluble UVA and UV-B filters known to the person skilled in the art. For example, the sunscreen formulation according to the invention can comprise:
paraminobenzoic acid (PABA) and derivatives thereof, such as dimethyl-, ethyldihydroxypropyl-, ethylhexyldimethyl-, ethyl-, glyceryl- and 4-bis-(polyethoxy)-PABA.
cinnamates, such as methylcinnamates and methoxycinnamates comprising octyl methoxycinnamate, ethyl methoxycinnamate, 2-ethylhexyl p-methoxycinnamate, isoamyl p-methoxycinnamate, diisopropyl cinnamate, 2-ethoxyethyl 4-methoxy-cinnamate, DEA methoxycinnamate (diethanolamine salt of p-methoxyhydroxycinnamate), diisopropyl methylcinnamate.
benzophenones, such as 2,4-dihydroxy-, 2-hydroxy-4-methoxy-, 2,2′-dihydroxy-4,4′-dimethoxy-, 2,2′-dihydroxy-4-methoxy-, 2,2′,4,4′-tetrahydroxy-, 2-hydroxy-4-methoxy-4′-methylbenzophenones, sodium 2,2′-dihydroxy-4,4′-dimethoxy-5-sulphobenzophenones.
dibenzoylmethanes, such as butylmethoxydibenzoylmethane, in particular 4-tert-butyl-4′-methoxydibenzoylmethane.
2-phenylbenzimidazole-5-sulphonic acid and phenyldibenzimidazolesulphonic acid esters and salts thereof.
diphenylacrylates, such as alkyl alpha-cyano-beta,beta-diphenylacrylates, such as octocrylene.
triazines, such as 2,4,6-trianilino-(p-carbo-2-ethylhexyl-1-oxy)-1,3,5-triazine, ethylhexyltriazone and diethylhexylbutamidotriazone.
camphor derivatives, such as 4-methylbenzylidene- and 3-benzylidenecamphor and terephthalylidenedicamphorsulphonic acid, benzylidenecamphorsulphonic acid, camphorbenzalkonium methosulphate and polyacrylamidomethylbenzylidenecamphor;
salicylates, such as dipropylene glycol, ethylene glycol, ethylhexyl, isopropylbenzyl, methyl, phenyl, 3,3,5-trimethyl and TEA salicylates (compound of 2-hydroxybenzoic acid and 2,2′,2″-nitrilotrisethanol);
esters of 2-aminobenzoic acid.

The sunscreen formulation can also comprise compounds known to the person skilled in the art, such as organic solvents, thickeners, emulsifiers, emollients, antifoams, antioxidants, plant extracts, moisturizing agents, perfumes, preservatives and/or dyes, complexing agents, anionic, cationic, nonionic or amphoteric polymers or mixtures thereof, propellant gases and finely divided powders, including metal oxide pigments with a particle size of from 100 nm to 20 μm.

Suitable emollients are, in particular, avocado oil, cotton seed oil, behenyl alcohol, butyl myristate, butyl stearate, cetyl alcohol, cetyl palmitate, decyl oleate, dimethyl polysiloxane, di-n-butyl sebacate, thistle oil, eicosanyl alcohol, glyceryl monoricinoleate, hexyl laurate, isobutyl palmitate, isocetyl alcohol, isocetyl stearate, isopropyl isostearate, isopropyl laurate, isopropyl linoleate, isopropyl myristate, isopropyl palmitate, isopropyl stearate, isostearic acid, cocoa butter, coconut oil, lanolin, lauryl lactate, corn oil, myristyl lactate, myristyl myristate, evening primrose oil, octadecan-2-ol, olive oil, palmitic acid, palm kernel oil, polyethylene glycol, rapeseed oil, castor oil, sesame oil, soya oil, sunflower oil, stearic acid, stearyl alcohol, triethylene glycol.

Suitable emulsifiers are, in particular, glycerol monolaurate, glycerol monooleate, glycerol monostearate, PEG 1000 dilaurate, PEG 1500 dioleate, PEG 200 dilaurate, PEG 200 monostearate, PEG 300 monooleate, PEG 400 dioleate, PEG 400 monooleate, PEG 400 monostearate, PEG 4000 monostearate, PEG 600 monooleate, polyoxyethylene(4) sorbitol monostearate, polyoxyethylene(10) cetyl ether, polyoxyethylene(10) monooleate, polyoxyethylene(10) stearyl ether, polyoxyethylene(12) lauryl ether, polyoxyethylene(14) laurate, polyoxyethylene(2) stearyl ether, polyoxyethylene(20) cetyl ether, polyoxyethylene(20) sorbitol monolaurate, polyoxyethylene(20) sorbitol monooleate, polyoxyethylene(20) sorbitol monopalmitate, polyoxyethylene(20) sorbitol monostearate, polyoxyethylene(20) sorbitol trioleate, polyoxyethylene(20) sorbitol tristearate, polyoxyethylene(20) stearyl ether, polyoxyethylene(23) lauryl ether, polyoxyethylene(25) oxypropylene monostearate, polyoxyethylene(3.5) nonylphenol, polyoxyethylene(4) lauryl ether, polyoxyethylene(4) sorbitol monolaurate, polyoxyethylene(5) monostearate, polyoxyethylene(5) sorbitol monooleate, polyoxyethylene(50) monostearate, polyoxyethylene(8) monostearate, polyoxyethylene(9.3) octylphenol, polyoxyethylene sorbitol lanolin derivatives, sorbitol monolaurate, sorbitol monooleate, sorbitol monopalmitate, sorbitol monostearate, sorbitol monostearate, sorbitol sesquioleate, sorbitol tristearate, sorbitol trioleate.

Suitable propellant gases may be propane, butane, isobutane, dimethyl ether and/or carbon dioxide.

Suitable finely divided powders may be chalk, talc, kaolin, colloidal silicon dioxide, sodium polyacrylate, tetraalkyl- and/or trialkylarylammonium smectites, magnesium aluminium silicates, montmorillonite, aluminium silicates, fumed silicon dioxide, fumed titanium dioxide.

Typically, the sunscreen composition according to the invention can be in the form of an emulsion (O/W, W/O or multiple), aqueous or aqueous-alcoholic gel or oil gel, and be supplied in the form of lotions, creams, milk sprays, mousse, as stick or in other customary forms.

### EXAMPLES

### Analysis

The BET surface area is determined in accordance with DIN 66131.

The average aggregate area, the average aggregate diameter (ECD) and the area ascertained by electron microscopy (EMSA) are determined by image analysis in accordance with ASTM 3849-89. In this method, the area of about 1500 aggregates is determined and the arithmetic mean is calculated from this. The image analyses were carried out using a TEM instrument from Hitachi H 7500 and a CCD camera MegaView II from SIS. The image magnification for the evaluation was 30 000:1 at a pixel density of 3.2 nm. The number of evaluated particles was greater than 1000. The preparation was carried out in accordance with ASTM 3849-89. The lower threshold value with regard to detection was 50 pixels.

Here, ECD (Equivalent Circle Diameter) means the diameter of a circle equal in area.

XPS-ESCA (XPS=X-ray Photoelectron Spectroscopy; ESCA=Electron Spectroscopy for Chemical Analysis): the evaluation of the XPS spectra is based on the general recommendations in DIN specialist report No. 39, the report DMA(A) 97 of the National Physics Laboratory, Teddington U.K. and the previous findings on the development-accompanying standardization of the working committee “Oberflächen-und Mikrobereichsanalysen” NMP816 (DIN). In addition, for the evaluation, the existing comparative spectra for the class of substance present in each case and corresponding results from the specialist literature are taken into consideration. The values are calculated by background subtraction, taking into consideration the relative sensitivity factors of the electron level stated in each case.

Photocatalytic activity: the degradation of the model noxious substance dichloroacetic acid is monitored by reference to the consumption of sodium hydroxide solution for keeping the pH constant. The following stoichiometry is known for the photocatalytic DCA degradation: CHCl₂CO₂⁶⁻+O₂→H⁺+2Cl⁻+2 CO₂. For this, the initial increase in the proton formation curves is used to firstly determine the degradation rate [nM/s] and, from this, the photon efficiency (in %) based on the incident light intensity. The photon efficiency is an absolute measure of the photocatalytic activity. It is calculated from the degradation rate based on the photon flow.

### Example 1

3 kg/h of zinc powder (particle size d₅₀=25 μm) are conveyed, by means of a stream of nitrogen (15 Nm³/h), to a vaporization zone where a hydrogen/air flame, hydrogen 14.5 Nm³/h, air 30 Nm³/h, burns. The zinc is vaporized here.

Vaporization zone conditions: lambda: 0.80, average residence time: 1009 ms, temperature: 1080° C.

Subsequently, 65 Nm³/h of oxidation air are added to the reaction mixture and then 2.45 kg/h of TEOS are introduced into the oxidation zone by means of 4 Nm³/h of atomization air.

Zinc oxidation zone conditions: lambda: 8.76; average residence time: 29 ms, temperature: 800° C.

Silicon oxidation zone conditions: lambda: 4.04; average residence time: 51 ms, temperature: 760° C.

To cool the hot reaction mixture, 200 Nm³/h of quenching air are added. The resulting powder is then separated off from the gas stream by filtration.

The powder has the physicochemical values shown in Table 2.

Examples 2 to 5 are carried out analogously to Example 1. Feed materials and amounts used are given in Table 1. The physicochemical values are given in Table 2.

Table 2, Example 6 (comparative example) likewise shows physicochemical values of commercially available zinc oxide particles coated with silicon dioxide (Showa Denko ZS-032).

### TEM Micrographs:

FIGS. 1A and 1B show TEM micrographs of the zinc silicon oxide particles according to the invention from Example 1 (FIG. 1A) and the commercially available particles of Example 6 (FIG. 1B). The two micrographs show the same magnification. The significantly smaller aggregate dimensions of the zinc silicon oxide particles according to the invention can be seen. This is also evident from the values in Table 2.

FIG. 2 shows a high-resolution TEM micrograph of a zinc silicon oxide particle according to the invention from Example 1. The core-shell structure can be clearly seen. The two areas labelled A (shell) and B (core) were analysed using EDX (energy-dispersive analysis of characteristic X-rays).

TABLE 1
Feed materials, amounts used and process parameters

| | | Example | | | | |
|---|---|---|---|---|---|---|
| | | 1 | 2 | 3 | 4 | 5 |
| Vaporization zone | | | | | | |
| Zinc | kg/h | 3 | 4 | 4 | 4 | 4 |
| Hydrogen | Nm³/h | 14.5 | 14.5 | 14.5 | 14.5 | 16.2 |
| Air | Nm³/h | 30 | 30 | 30 | 30 | 34 |
| Nitrogen | Nm³/h | 15 | 15 | 15 | 15 | 14 |
| Lambda | | 0.80 | 0.78 | 0.78 | 0.78 | 0.80 |
| Temperature | ° C. | 1080 | 1050 | 1050 | 1050 | 1100 |
| Average residence time | Ms | 1009 | 1026 | 1026 | 1026 | 923 |
| Zn oxidation zone | | | | | | |
| Air | Nm³/h | 65 | 80 | 65 | 65 | 80 |
| Lambda | | 8.76 | 9.71 | 7.89 | 7.89 | 9.59 |
| Temperature | ° C. | 800 | 820 | 800 | 810 | 920 |
| Average residence time | Ms | 29 | 41 | 48 | 47 | 37 |
| Si oxidation zone | | | | | | |
| Si precursor | | Si(OEt)₄ | Si(OEt)₄ | Si(OEt)₄ | MeSi(OEt)₃ | Si(OEt)₄ |
| Si precursor | kg/h | 2.45 | 1.37 | 2.45 | 2 | 2.45 |
| Atomization air | Nm³/h | 4 | 4 | 4 | 4 | 4 |
| Lambda | | 4.04 | 8.88 | 3.98 | 4.56 | 4.96 |
| Temperature | ° C. | 760 | 610 | 780 | 750 | 820 |
| Average residence time | ms | 51 | 38 | 36 | 37 | 30 |
| Quenching zone | | | | | | |
| Air | Nm³/h | 200 | 200 | 250 | 220 | 300 |

TABLE 2
Physicochemical properties of the zinc silicon oxide powders

| | | Example | | | | | |
|---|---|---|---|---|---|---|---|
| | | | | | | | 6** |
| | | 1 | 2 | 3 | 4 | 5 | (comparison) |
| Zn/Si | at %/at % | 9.1 | 9.3 | 5.2 | 5.4 | 5.2 | 3.7 |
| Sum of Zn + Si + O* | | >99.7 | >99.7 | >99.7 | >99.7 | >99.7 | >99.7 |
| BET surface area | m²/g | 35 | 29 | 32 | 35 | 25 | 32 |
| Weight-averaged | nm | 23 | 24 | n.d. | n.d. | 35 | 100 |
| primary particle | | | | | | | |
| diameter | | | | | | | |
| Average aggregate | nm² | 1244 | 12302 | n.d. | n.d. | 33712 | 64819 |
| area | | | | | | | |
| Average aggregate | nm | 105 | 110 | n.d. | n.d. | 170 | 212 |
| diameter (ECD) | | | | | | | |
| Shell | nm | 2.1 | 1.2 | 1.8 | 1.6 | 2.3 | 2.4 |
| Carbon content | % by wt | 0.15 | 0.15 | 0.1 | 0.1 | 0.1 | 0.1 |
| UV max. absorption | nm | 369 | 367 | 367 | 369 | 369 | 372 |
| Transparency | | 4.5 | 5.0 | 4.4 | 5.1 | 4.0 | 2.7 |

*Calculated from ZnO and SiO₂, determined by X-ray fluorescence analysis;
n.d. = not determined;
**Comparison material: Showa Denko ZS-032.

The analysis does not permit a quantitative statement, but reveals a shell with silicon as main constituent in addition to a small amount of zinc. In the case of the core, as a consequence of the EDX analysis, besides the main constituent zinc, small fractions of silicon are also recorded.

Determination of the lattice distances in the high-resolution TEM spectra of zinc silicon oxide particles according to the invention clearly shows that the core consists of zinc oxide.

### X-Ray Diffraction Spectrum:

FIG. 3 shows the X-ray diffraction spectrum of the zinc silicon oxide particles according to the invention from Example 1. The pulses are plotted against the position 2 theta in degrees. Besides the typical pulses which can be assigned to zinc oxide, in this example, pulses of low intensity are detected at 2 theta=21.89, 24.73 and 26.78.

### UV Absorption/Transparency:

FIG. 4 shows the UV spectrum of the zinc silicon oxide particles from Example 1 (conditions: 0.05% by wt, path length: 1 mm). Here, the absorbance is plotted against the wavelength in nm. The maximum absorbance is 0.7 at a wavelength of 368 nm. The absorbance at the wavelength 450 nm is 0.155. The transparency is 4.5, expressed as absorbance at 368 nm divided by absorbance at 450 nm. The transparency is accordingly significantly higher than in the case of the commercially available material (Example 6) with 2.7.

### Differential Calorimetry (DSC):

FIG. 5A shows the DSC of the zinc silicon oxide particles according to the invention from Example 1 (thick solid line), of the commercially available particles from Example 6 ( - - - ) and of other commercially available zinc oxide particles coated with silicon dioxide (Finex 50, Sakai; [symbol] ). Here, μV/mg (y-axis) is plotted against the temperature (° C.).

FIG. 5B shows the mass loss of these particles upon heating. In contrast to the commercially available particles, in the case of the particles according to the invention, phase conversions are only evident to a small extent and the mass loss is only slight.

### Photocatalytic Activity:

Over a period of 6 hours, no degradation of DCA is reproducibly detectable. The oxide powder from Example 1 on which the dispersion is based has no photocatalytic activity.

### Example 7

### Preparation of a Dispersion According to the Invention

The zinc silicon oxide particles from Example 1 are added in portions, with stirring, to 50 g of water to which 0.1% by weight of polyacrylic acid in the form of the sodium salt has been added, until a solids content of 10% by weight results. Then, the mixture is dispersed in each case for one minute using an ultrasound finger (diameter: 7 mm, instrument: ultrasound processor UP 400s, power: 400 W, Dr Hielscher).

### Example 8

### Preparation of a Coating Preparation According to the Invention Based on Acrylic/Polyurethane

The dispersion from Example 7 is added, under dispersing conditions, to a standard commercial acrylic/polyurethane binder preparation (Relius Aqua Siegel Gloss) so that a coating preparation with a content of composite particles of 2% by weight results.

### Example 9

### Preparation of an Acrylic-Based Coating Preparation According to the Invention

Procedure as in Example 8, but using a standard commercial acrylic binder preparation. (Macrynal SM 510 (Cytec), Desmodur N75 (Bayer)).

### Example 10

### UV Resistance Upon Coating Wood

The coating preparations from Example 8 and 9 are each used to coat 3 pinewood samples which have been pretreated with a primer (Relius Aqua Holz Grund) (QUV-B 313; DIN EN 927-6, ISO 11507, ASTM D 4857). The comparison used is pinewood samples coated with a coating preparation which is free from composite particles, acrylic/polyurethane-based (Relius Aqua Siegel Gloss).

After a test time of 1000 hours, the coatings from Examples 8 and 9 exhibit significantly lower yellowing, considerably higher gloss and no brittleness or cracks in the coating compared to the coating without the zinc silicon oxide particles according to the invention.

### Example 11

### Sunscreen Formulation

The formulation below was used to prepare a sunscreen composition containing 4% by weight of the zinc silicon oxide particles according to the invention as in Example 1.

| Phase | Constituent | % by weight |
|---|---|---|
| A | Isolan GI 34 | 3.0 |
| | Castor oil | 1.2 |
| | Tegesoft OP | 10.0 |
| | Tegesoft Liquid | 5.0 |
| | Glycerol 86% | 3.0 |
| B | Paracera W80 | 1.8 |
| | Isohexadecane | 5.0 |
| C | Zinc silicon oxide particles | 4.0 |
| D | Magnesium sulphate | 0.5 |
| | Demineralized water | 66.5 |

Phase A is heated to 70° C. in a mixer. After melting on a magnetic heating plate at 80° C., phase B is added to phase A. Phase C is stirred into the oil phase at about 300 rpm and under reduced pressure. Phase D is likewise heated to 70° C. and added to the mixture of A-C under reduced pressure.

1. Zinc silicon oxide particles with a core-shell structure, wherein
they have a Zn/Si ratio of from 2 to 75, in atom %/atom %,
the fraction of Zn, Si and O is at least 99% by weight, based on the zinc silicon oxide particles,
they have a
BET surface area of from 10 to 60 m²/g,
a weight-averaged primary particle diameter of from 10 to 75 nm
an average aggregate area of less than 40 000 nm² and
an average aggregate diameter (ECD) of less than 200 nm,
the core is crystalline and consists of aggregated primary particles of zinc oxide and
the shell surrounds the aggregated zinc oxide primary particles and consists of one or more compounds containing the elements Si and O.

2. Zinc silicon oxide particles according to claim 1, wherein the shell consists of one or more compounds containing the elements Si, O and Zn.

3. Zinc silicon oxide particles according to claim 1, wherein the shell is amorphous.

4. Zinc silicon oxide particles according to claim 1, wherein the Zn/Si ratio is 3 to 15.

5. Zinc silicon oxide particles according to claim 1, wherein the BET surface area is 20 to 40 m²/g.

6. Zinc silicon oxide particles according to claim 1, wherein the average aggregate area is less than 20 000 nm² and the average aggregate diameter is less than 150 nm.

7. Zinc silicon oxide particles according to claim 1, wherein the thickness of the shell is 0.1 to 10 nm.

8. Zinc silicon oxide particles according to claim 1, wherein they have a ratio of maximum absorbance/absorbance at 450 nm of from 4 to 8.

9. Zinc silicon oxide particles according to claim 1, wherein they have a photocatalytic activity, expressed by the photon efficiency and determined by the degradation of dichloroacetic acid, of less than 0.4.

10. Zinc silicon oxide particles according to claim 1, wherein, upon heating to 1400° C., they lose less than 2% of their mass, and phase conversions occur only to a slight extent.

11. Zinc silicon oxide particles according to claim 1, wherein they comprise at most 0.2% by weight of carbon.

12. Zinc silicon oxide particles according to claim 1, wherein they have at most 20 ppm of Pb, at most 3 ppm of As, at most 15 ppm of Cd, at most 200 ppm of Fe, at most 1 ppm of Sb and at most 1 ppm of Hg.

13. Process for producing zinc silicon oxide particles according to claim 1, wherein a mixture of zinc vapour and a hydrogen-containing combustion gas is passed to an oxidation zone of a reactor, where it is reacted with an oxygen-containing gas and at least one organic silicon compound at temperatures of from 500 to 1500° C., then the reaction mixture is cooled and the pulverulent solids are separated off from gaseous substances, where
the silicon compound is chosen from at least one compound from the group comprising
R′ₓSi(OR)₄₋ₓ, where R=Me, Et; R′=H, Me, Et; x=0-4,
R″ᵤMeᵥSiOSiMeᵥR″ᵤ where R″=H, Et; u=0, 1, 2; v=1, 2, 3; u+v=3;
R′″₄Si where R′″=H, Me, Et and/or cyclic polysiloxanes (R″″MeSiO)y where R″″=H, Me, Et, y=3-5,
the oxygen fraction of the oxygen-containing gas is at least sufficient to completely oxidize zinc, the organic silicon compound and the hydrogen
the average residence time of the reactants in the oxidation zone is 5 ms to 30 s.

14. Process according to claim 13, wherein the silicon compound is tetraethoxysilane and/or tetramethoxysilane.

15. Process according to claim 13, wherein the temperatures required for the oxidation and optionally vaporization are provided by a flame which is formed by igniting a hydrogen-containing combustion gas with an oxygen-containing gas, where in the oxidation zone 1<lambda≦10.

16. Process according to claim 15, wherein the vaporization zone, 0.5≦lambda≦1.

17. Dispersion comprising the zinc silicon oxide particles according to claim 1.

18. Coating composition comprising the zinc silicon oxide particles according to claim 1 and at least one binder.

19. Sunscreen formulation comprising the zinc silicon oxide particles or dispersion.

20. A coating composition comprising zinc silicon oxide dispersion according to claim 17.
